(19) United States
(12) Patent Application Publication
(10) Pub. No.: US 2021/0007788 A1
(43) Pub. Date: Jan. 14, 2021

(54) END EFFECTOR ENABLING GRASPING OF TISSUE AND PLASMA RADIATION TO TISSUE, AND ENDOSCOPIC SYSTEM COMPRISING SAID END EFFECTOR

(71) Applicant: TOKYO INSTITUTE OF TECHNOLOGY, Tokyo (JP)

(72) Inventors: Akitoshi OKINO, Tokyo (JP); Hidekazu MIYAHARA, Tokyo (JP); Hiroaki KAWANO, Tokyo (JP); Yuta HAYASHI, Tokyo (JP); Yuma SUENAGA, Tokyo (JP); Toshihiro TAKAMATSU, Hyogo (JP); Manabu KUROSAWA, Hyogo (JP)

(21) Appl. No.: 17/041,156

(22) PCT Filed: Mar. 25, 2019
(86) PCT No.: PCT/JP2019/012448

§ 371 Date: Sep. 24, 2020

(30) Foreign Application Priority Data
Mar. 26, 2018 (JP) 2018-057947

(51) Int. Cl. A61B 18/04; A61B 18/08; A61B 18/12; A61B 1/018

(52) U.S. Cl. CPC A61B 18/042; A61B 18/085; A61B 2018/00589; A61B 1/018; A61B 18/12

(57) ABSTRACT

Provided is an end effector enabling the grasping of tissue and plasma radiation to tissue. This end effector comprises: a grasping member for grasping tissue; and a plasma generation mechanism capable of generating plasma. A pulling means is connected to the plasma generation mechanism, and by operation of the connected pulling means, grasping of tissue by the grasping means is achieved. The plasma generation means is configured so as to enable plasma to be radiated at the position where the grasping member grasps tissue.

### TECHNICAL FIELD

The present invention relates to an end effector that enables grasping of tissue and plasma irradiation to tissue, and an endoscopic system comprising the end effector.

### BACKGROUND ART

A low temperature plasma generation apparatus has been conventionally known (see, for example, Non Patent Literature 1). Aside from surface processing, low temperature plasma can obtain effects such as sterilization, blood coagulation (hemostasis) and wound healing in the medical field. In particular, application to hemostasis is expected because low temperature plasma can coagulate blood in a short time period without damaging tissue.

### CITATION LIST

### Non Patent Literature

[NPL 1] Mynavi Corporation, “Tokodai nado, −90 ˜+150° C. de ondo wo seimitsu seigyo kanou na taikiatsu purazuma souchi wo kaihatsu (Tokyo Institute of Technology and others developed an atmospheric pressure plasma apparatus that can precisely control temperature between −90 and +150° C.)”, [online], [searched on Jan. 31, 2018], internet, <URL:https://news.mynavi.jp/article/20111026-a080/>

### SUMMARY OF INVENTION

### Technical Problem

However, low temperature plasma was limited in terms of hemostasis effect against an exposed blood vessel or spurting bleeding.

The present invention was invented in view of the problem discussed above, wherein the purpose is to provide an improved end effector for hemostasis and an endoscopic system comprising the end effector.

### Solution to Problem

In one aspect of the present invention, the end effector of the present invention comprises: a grasping member for grasping tissue; and a plasma generation mechanism that can generate plasma.

In one embodiment of the present invention, the end effector may further comprise a hinge part, wherein: the grasping member and the plasma generation mechanism may be connected to each other at the hinge part; and the grasping member may be configured to be able to rotate around the hinge part.

In one embodiment of the present invention, the end effector may further comprise a connection part that can connect with a pulling means that can pull the plasma generation mechanism, wherein activation of the pulling means that has been connected may achieve grasping of the tissue with the grasping member.

In one embodiment of the present invention, the connection part may be configured so that the puling means would be detachable.

In one embodiment of the present invention, the grasping member may be configured to be able to be electrically controlled.

In one embodiment of the present invention, the plasma generation mechanism may be configured so that the plasma can be irradiated to a position where the grasping member grasps the tissue.

In one embodiment of the present invention, the grasping member may comprise a plurality of grasping pieces.

In one embodiment of the present invention: the plasma generation configuration may have a housing shape having a hollow part; the housing may comprise a first electrode and a second electrode that is different from the first electrode; and the plasma generation mechanism may turn gas that passes through the hollow part into plasma by electric release between the first electrode and the second electrode.

In one aspect of the present invention, the endoscopic system of the present invention comprises the end effector of any one of claims 1 to 8.

In one embodiment of the present invention, the endoscopic system may further comprise: a gas supply source that can supply gas that is to be turned into plasma with the plasma generation mechanism, wherein the gas supply source can supply one or more types of gas; a power source that can switch among a plurality of modes; and a pulling means that can be connected to the connection part of the end effector.

In one embodiment of the present invention, the plurality of modes may include at least two of a low temperature plasma mode, an APC (argon plasma coagulation) mode and a high frequency coagulation mode.

In one embodiment of the present invention, the endoscopic system may further comprise a pulse gas system for enabling pulse-like supply of gas that is to be turned into plasma to the hollow part.

### Advantageous Effects of Invention

According to the present invention, it is possible to provide an improved end effector for hemostasis and an endoscopic system comprising the end effector.

### BRIEF DESCRIPTION OF DRAWINGS

FIG. 1 shows a schematic drawing of one example of an endoscopic system 10 comprising the end effector of the present invention.

FIG. 2 is a cross-section drawing showing one example of the configuration of the end effector 100 of the present invention.

FIG. 3 is a cross-section drawing showing the end effector 100 of FIG. 2.

### DESCRIPTION OF EMBODIMENTS

Herein, the term “distal” refers to a portion that is farther away from a user (operator) and the term “proximal” refers to a portion that is closer to the user. Herein, “about” refers to the concept of being in the range of ±10% of the number that follows thereafter.

The embodiment of the present invention is explained below while referring to the drawings. Furthermore, the same reference number is used for the same component throughout herein.

The present invention is characterized by an end effector that enables grasping of tissue and plasma irradiation to tissue and an endoscopic system comprising the end effector and an operation part. Movement of an end effector (for example, grasping of tissue or plasma irradiation to tissue) can be controlled by the operation part.

A power source having one or more modes and a supply source for supplying a supply can be connected to the operation part as needed. In a preferable embodiment, the power source can have a plurality of modes. While these plurality of modes may typically include two or preferably three of a low temperature plasma mode, an APC (argon plasma coagulation) mode and a high frequency coagulation mode, the present invention is not limited thereto. One example of the supply source is a gas supply source for supplying gas that is to be turned into plasma. Gas is supplied from a gas supply source to an end effector through an operation part. The type of gas to be supplied can be differentiated in accordance with one or more mode of the power source. The gas supplied to the end effector will be turned into plasma in the end effector and will be irradiated to tissue. This leads to treatment (for example, hemostasis or sterilization) of the tissue. In a preferable embodiment, a gas supply source may comprise a pulse gas system for enabling pulse-like supply of gas. A pulse gas system can clarify a target for hemostasis by pulse-like firing of gas. Furthermore, treatment (for example, hemostasis or ligation) of tissue is carried out by the end effector grasping the tissue.

A preferable embodiment of the end effector and the endoscopic system of the present invention is explained below.

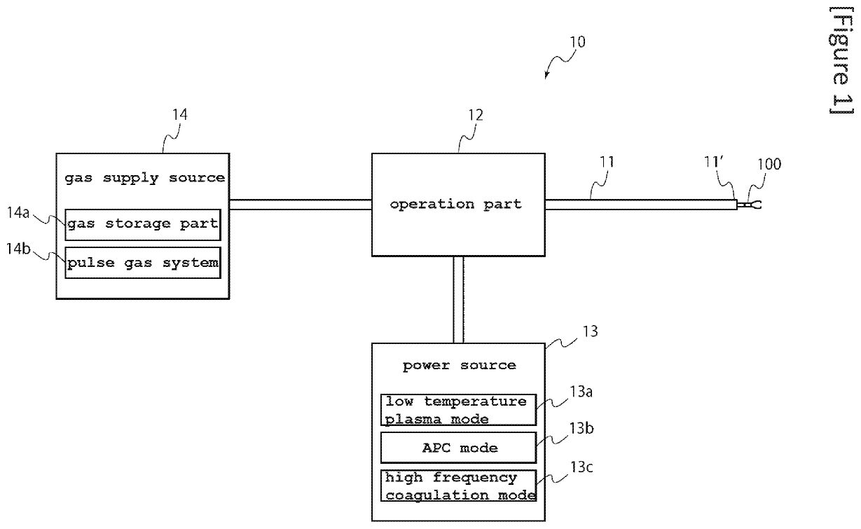

FIG. 1 shows a schematic drawing of one example of an endoscopic system 10 comprising the end effector of the present invention.

An endoscopic system 10 comprises an insertion part 11, an operation part 12 connected to a proximal end part of the insertion part 11 and a power source 13 and a gas supply source 14 connected to the operation part 12.

The gas supply source 14 is for supplying gas that is to be turned into plasma. The gas supply source 14 is interconnected with the operation part 12 as shown in FIG. 1, and thereby supplies gas to the operation part 12 and eventually to the insertion part 11. The gas supply source 14 comprises a gas storage part 14a for storing gas and a pulse gas system 14b for enabling pulse-like supply of gas.

The gas storage part 14a is configured so as to enable storage of a plurality of types of gas and extraction of stored gas. The form of storage of a plurality of types of gas is arbitrary. For example, a plurality of types of gas may be stored by comprising a plurality of gas tanks that each can store one type of gas, each type of gas may be stored within each interior space of a housing that has a plurality of divided spaces inside, or a plurality of types of gas may be stored so as to enable extraction of each type of gas as needed in a state in which a plurality of types of gas are mixed. In addition, the gas stored in the gas storage part 14a is, for example, but not limited to, argon, carbon dioxide, oxygen, nitrogen, helium and air.

The pulse gas system 14b is for enabling pulse-like supply of gas that is to be turned into plasma which is supplied from the gas supply source 14 to the insertion part 11 through the operation part 12. The pulse gas system 14b is configured to be able to provide a high-speed air stream with a predetermined pressure to gas that is to be turned into plasma in a predetermined time interval, and thereby enables pulse-like gas supply to the insertion part 11 through the operation part 12. The pulse gas system 14b is configured so as to enable addition of a high-speed air stream to gas that is to be turned into plasma. The pressure, interval between additions and number of irradiations of the air stream that is to be added by the pulse gas system 14b can be appropriately adjusted in accordance with a condition required for treatment of tissue or the like. The pressure of the air stream is, for example, about 0.3 to about 0.9 MPa. In addition, the interval between additions is, for example, about 0.1 to about 5 seconds. Furthermore, the number of irradiations is, for example, about 5 to about 20 times. In one embodiment, a high-speed air stream may be added 5 times, 10 times, or times, under the condition of being a high-speed air stream with the pressure of 0.3 MPa with the interval of 0.1 second. In other embodiment, a high-speed air stream may be added 5 times, 10 times, or 20 times, under the condition of being a high-speed air stream with the pressure of 0.6 MPa with the interval of 0.1 second. In yet other embodiment, a high-speed air stream may be added 5 times, 10 times, or 20 times, under the condition of being a high-speed air stream with the pressure of 0.9 MPa with the interval of 0.1 second. However, the present invention is not limited thereto. As such, the technique of using gas that is to be turned into plasma in a form of a pulse to irradiate the plasma to a lesion enables the pulse-like plasma to treat the lesion while a high-speed air stream blows off obstructive blood, foreign objects and the like.

Thus, pulse-like gas irradiation contributes to improvement of visibility and high-speed hemostasis of a lesion.

While the number of the gas supply source 14 is one in the example shown in FIG. 1, the present invention is not limited thereto. The number of the gas supply source 14 is any number that is one or greater. For example, a carbon dioxide supply source for storing and supplying carbon dioxide alone and an argon supply source for storing and supplying argon alone may be interconnected with the operation part 12.

Furthermore, a supply source (not shown) for supplying a supply other than gas may be further connected to the operation part 12. The supply supplied from the supply source (not shown) may be, for example, an illumination light that illuminates the periphery of a therapy site or an examination site, a laser light source for guiding an irradiation position of plasma irradiated from the end effector, or a cooling water that cleans a therapy site and cools the endoscope or the like.

The power source 13 is for supplying necessary power to the operation part 12 and eventually to a device incorporated in the insertion part 11. The power source 13 is configured be switchable among a plurality of modes. In the example shown in FIG. 1, the power source 13 is switchable among a low temperature plasma mode 13a, an APC (argon plasma coagulation) mode 13b and a high frequency coagulation mode 13c. However, the present invention is not limited thereto. For example, the switching may be between the low temperature plasma mode 13a and the APC (argon plasma coagulation) mode 13b, may be between the low temperature plasma mode 13a and the high frequency coagulation mode 13c, or may be between the APC (argon plasma coagulation) mode 13b and the high frequency coagulation mode 13c.

The low temperature plasma mode 13a is a mode for carrying out plasma irradiation at a low temperature (for example, about −90 to about 160° C., more preferably, about to about 100° C.). The use of plasma at 40° C. to about 100° C. is preferable in terms of being able to dehydrate blood or the like with heat while reducing thermal damage in addition to the chemical blood coagulation effect. An end effector 100 can turn gas supplied from the gas supply source 14 into plasma at a low temperature and irradiate the plasma to a lesion at a low temperature as discussed below by switching the power source 13 to the low temperature plasma mode 13a. One example of the gas that is to be turned into plasma at a low temperature is as previously mentioned as an example of gas stored in the gas storage part 14a. The safety of low temperature plasma is high. In addition, high hemostasis effect is obtained for gushing bleeding. However, effect on hemostasis of spurting bleeding, exposed blood vessel, or the like is limited.

The APC mode 13b is a mode for realizing treatment of a lesion using APC. The power source 13 applies high frequency current to argon supplied from the gas supply source 14 by switching the power source 13 to the APC mode 13b. This enables treatment of the lesion using APC. APC achieves high hemostasis effect on gushing bleeding. This is because APC treats gushing bleeding by cauterizing a wide range with large surface area cauterized by plasma gas and not by carrying out a local cauterization with a small cauterization surface area. However, since APC does not have a grasping structure that can seal a bleeding part of a blood vessel with thermal denaturation, the hemostasis effect on spurting bleeding and an exposed blood vessel is low.

The high frequency coagulation mode 13c is a mode for realizing cauterization of a lesion using a high frequency current. A high frequency current can be applied to the end effector 100 to flow to a lesion and coagulate the lesion with heat caused by the high frequency current by switching the power source 13 to the high frequency coagulation mode 13c. The frequency of the high frequency applied in the high frequency coagulation mode 13c may be, for example, 10 kHz to 5 MHz, preferably, 10 kHz to 1 MHz, more preferably, 10 kHz to 500 kHz. High frequency coagulation achieves high hemostasis effect on various states of bleeding such as spurting bleeding. However, high frequency coagulation may cause damage to tissue.

The operation part 12 is for operating the insertion part 11 and a device incorporated in the insertion part 11. The operation part 12 is interconnected with the supply source 13 as shown in FIG. 1 and is configured to be able to control the amount of supply supplied from the operation part 12. In addition, the operation part 12 is interconnected with the power source 13 as shown in FIG. 1 and is configured to be able to control the switching of the power source 13 among a plurality of modes.

The insertion part 11 is a portion inserted in a body. The insertion part 11 is configured to be controlled by the operation part 12 and to be able to bend to change the direction of the insertion part 11 in accordance with the input in the operation part 12. The insertion part 11 comprises the end effector 100 that can project from a distal end part 11′ of the insertion part 11. The size of the diameter of the insertion part 11 can be any size, preferably as small as possible so as to be movable inside a very small space (for example, inside intestines or inside digestive organs). For example, when an endoscope 10 is an endoscope for a large intestine, the size would be about 13 mm. However, the present invention is not limited thereto. In addition, the size of the diameter of the end effector can be any size, preferably as small as possible so as to be movable inside a very small space (for example, inside intestines or inside digestive organs). For example, when the end effector is provided in a forceps channel of an endoscope for large intestines, the size would be about 3 mm. However, the present invention is not limited thereto.

The insertion part 11 comprises a channel for forceps and the end effector 100 is configured to be capable of going through the channel for forceps in accordance with the circumstance and projecting from an open end part of the channel for forceps on the distal end part 11′ of the insertion part 11. For example, the end effector will project when applying therapy to a therapy site using the end effector, and the end effector will be stored in the insertion part 11 when moving the endoscope 10 itself or the like.

The device incorporated in the insertion part 11 may comprise, for example, an imaging unit (for example, a camera lens) and a device for illumination (for example, a light) in addition to the end effector 100. The device incorporated in the insertion part 11 may be one or more. Furthermore, a nozzle for releasing a supply from the supply source 13 may be provided in the insertion part 11.

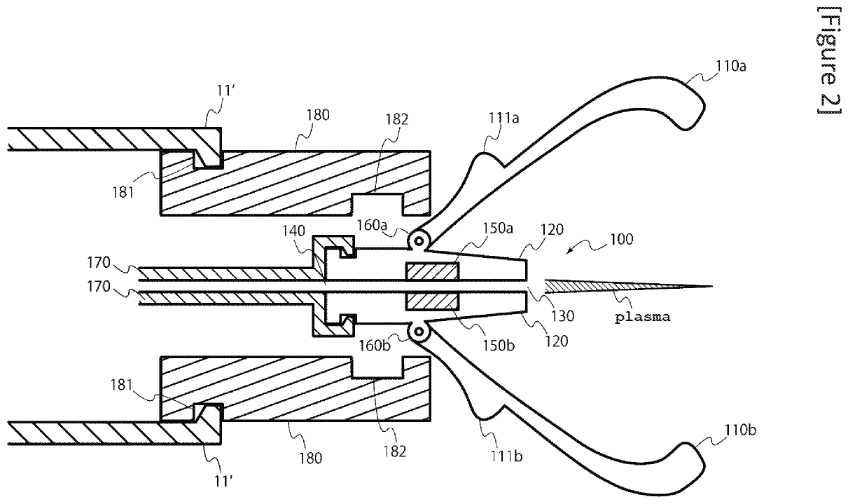

FIG. 2 is a cross-section drawing showing one example of a configuration of the end effector 100 of the present invention.

The end effector 100 of the present invention comprises a grasping member 110 for grasping tissue and a plasma generation mechanism 120 that can generate plasma.

In the example shown in FIG. 2, the grasping member 110 comprises a first grasping piece 110a and a second grasping piece 110b. The grasping member 110 shown in FIG. 2 is in an open state. Grasping of tissue is achieved by the first grasping piece 110a and the second grasping piece 110b working together. One example of the grasping member 110 is, but not limited to, a medical clip.

In a preferable example shown in FIG. 2, the grasping member 110 may comprise a projection part 111a on the first grasping piece 110a and comprise a projection part 111b on the second grasping piece 110b. The projection part 111a and the projection part 111b are used to maintain a closed state of the grasping member 110 as discussed in detail below. In addition, the projection part 111a and the projection part 111b play a role as a stopper mechanism so that the end effector 100 would not be overly pulled inside the insertion part 11.

The plasma generation mechanism 120 has a housing shape having a hollow part. The hollow part is defined between a release hole 130 on the distal end part of the plasma generation mechanism 120 and an inflow hole 140 on a proximal end part of the plasma generation mechanism 120. Gas supplied from the gas supply source 14 enters into the hollow part from the inflow hole 140 and passes through the hollow part to be released from the release hole 130. Since there may be a case in which high frequency is generated when the housing or the grasping member touches plasma, it is preferable that a portion where high frequency flow is not intended is insulative (for example, said portion may be configured with an insulative member, or may be coated with an insulative member such as resin or ceramic).

The plasma generation mechanism 120 comprises a first electrode 150a and a second electrode 150b as means for generating plasma. The first electrode 150a and the second electrode 150b are disposed, for example, along the interior wall of the hollow part so as not to interfere with the flow of gas. In the example shown in FIG. 2, the first electrode 150a and the second electrode 150b is embedded in the plasma generation mechanism 120 along the interior wall of the hollow part. For example, the first electrode 150a is an earthed electrode and the second electrode 150b is a high voltage electrode having voltage higher than the first electrode 150a. Alternatively, the first electrode 150a may be an electrode having voltage lower than the second electrode 150b.

When voltage is applied between the first electrode 150a and the second electrode 150b by a power source (not shown), electric discharge is generated between the first electrode 150a and the second electrode 150b. Therefore, gas that entered inside through the inflow hole 140 passes through the hollow part of the end effector 100 to be turned into plasma between the first electrode 150a and the second electrode 150b by the electric release between the first electrode 150a and the second electrode 150b to be released from the release hole 130. This causes the plasma to be ejected from the release hole 130, and irradiation of the plasma to an irradiation target (for example, bleeding site) causes blood coagulation and sterilization effect. The flow rate of the plasma generated by electric release between the first electrode 150a and the second electrode 150b is about over 0 to about 15 L/min, more preferably about over 0 to about 3 L/min. A low dose such as about over 0 to about 3 L/min may be preferable in terms of reducing generation of submucosal emphysema. Furthermore, the inflow hole 140 and the release hole 130 will have any shape as long as plasma can pass through. For example, the shape of the inflow hole 140 and the release hole 130 may be a circle, may be a square, or may be a polygon. The first electrode 150a and the second electrode 150b may be used in combination in the low temperature plasma mode, the APC mode and the high frequency coagulation mode, or different electrodes may be used for each mode.

While FIG. 2 exemplified a configuration that generates plasma by electric release generated between a first earthed electrode and a second electrode with higher voltage, the present invention is not limited thereto. For example, a bipolar-type configuration that generates plasma by earthing or applying low voltage to a plasma generation mechanism while applying high voltage to a pair of electrodes (i.e., first electrode and second electrode) may be realized, or a monopolar-type configuration that uses a counter electrode plate by not connecting a plasma generation configuration to a circuit while applying high frequency only to a pair of electrodes may be realized. Furthermore, there may be a configuration that is switchable between the bipolar-type configuration and the monopolar-type configuration by rendering the plasma generation mechanism to be switchable between an earthed or low voltage state and a state of not being connected to a circuit.

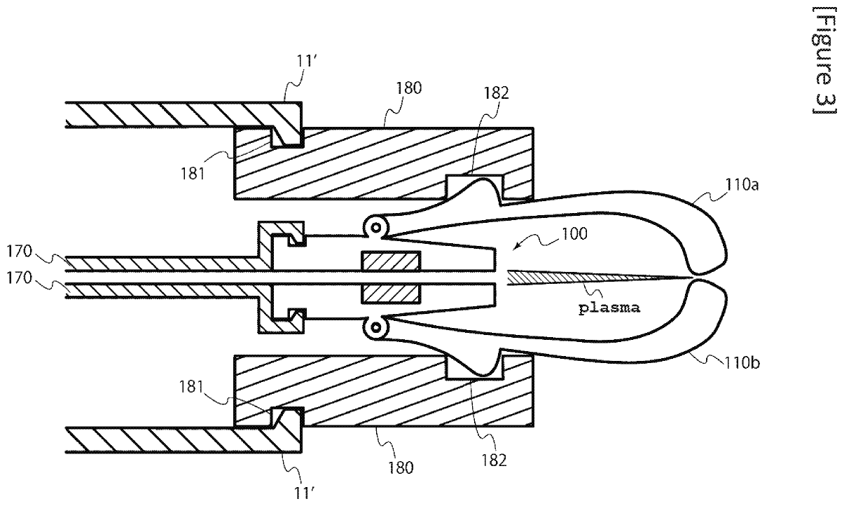

The end effector 100 further comprises a hinge part 160. In the example shown in FIG. 3, the hinge part 160 is provided to the plasma generation mechanism 120, wherein the hinge part 160 comprises a hinge part 160a for linking the grasping piece 110a to the plasma generation mechanism 120 and a hinge part 160b for linking the grasping piece 110b to the plasma generation mechanism 120. The grasping piece 110a is configured to be able to rotate around the hinge part 160a and the grasping piece 110b is configured to be able to rotate around the hinge part 160b. Therefore, the grasping member 110 can achieve opening/closing of the grasping member 110 by the rotational motion around the hinge part 160.

The endoscopic system 10 further comprises a pulling means 170 that can pull the plasma generation mechanism 120, wherein the pulling means 170 and the end effector 100 are configured to be able to be connected to each other. In other words, the end effector 100 plays a role of a connection part to connect with the pulling means 170.

Furthermore, the end effector 100 is configured to be detachable from the pulling means 170. In the preferable example shown in FIG. 2, the pulling means 170 comprises a convex part at the distal end part of the pulling means 170, wherein the convex part and a concave part comprised by the end effector 100 fit for connection with the end effector 100. The fitting strength of the convex part of the pulling means 170 and the concave part of the end effector are set to be weaker than the fitting strength of the concave part 181 of the lock mechanism 180 and the projection parts 111a, 111b of the grasping member 110 discussed below.

The pulling means 170 is configured to pull in the end effector 100 towards the direction of being pulled inside the insertion part 11 with a force equal to or stronger than the fitting strength of the convex part of the pulling means 170 and the concave part of the end effector 100 so that the fitting of the convex part of the pulling means 170 and the concave part of the end effector 100 would come undone to enable removal from the end effector 100.

In the preferable example shown in FIG. 2, the endoscopic system 10 further comprises a lock mechanism 180. The lock mechanism 180 is connected to the distal end part 11′ of the insertion part 11, and in the example shown in FIG. 2, the lock mechanism 180 comprises a concave part 181, wherein the concave part 181 fits with the convex part disposed in the distal end part 11′ of the insertion part 11 to achieve connection to the distal end part 11′ of the insertion part 11. The fitting strength of the convex part of the distal end part 11′ of the insertion part and the concave part 181 of the lock mechanism 180 is set to be weaker than the fitting strength of the concave part 182 of the lock mechanism 180 and the projection parts 111a, 111b of the grasping member 110 which is discussed below and substantially equal to the fitting strength of the convex part of the pulling means 170 and the concave part of the end effector 100.

The insertion part 11 is configured to pull in the insertion part towards the direction of being pulled inside the insertion part 11 with a force equal to or stronger than the fitting strength of the convex part of the distal end part 11′ of the insertion part and the concave part 181 of lock mechanism 180 so that the fitting of the convex part and the concave part would come undone to enable the insertion part 11 to be removed from the lock mechanism 180.

The lock mechanism 180 is configured to be able to maintain a closed state of the grasping member 110. In the example shown in FIG. 2, the lock mechanism 180 comprises a concave part 182 on the interior surface of the lock mechanism 180, wherein the projection part 111a and the projection part 111b engage with the concave part 182 to secure the rotational movement of the grasping member 110 and maintain the closed state of the grasping member 110.

The grasping member 110 is in an open state as shown in FIG. 2 in a normal state due to the force of a tension spring (not shown). When the pulling means 170 is pulled towards the direction of pulling the end effector 100 inside the insertion part 11 in such an open state, the end effector 100 starts to move towards the inside of the insertion part 11, and when the pulling means 170 is continued to be pulled towards the same direction, the grasping member 110 physically contacts the distal end part 11′ of the insertion part 11. When the pulling means 170 is pulled further towards the same direction, the grasping member 110 rotates around the hinge part 160 and shifts from an open state to a closed state to enable grasping of tissue.

The present embodiment explained a case in which the grasping member 110 is released in a normal state due to a spring or the like and the movement of the pulling means closes the grasping member 110. However, the present invention is not limited thereto. For example, the grasping member 110 may be closed in a normal state due to the force of a compression spring or the like, and the grasping member 110 may be opened by the movement of the pulling means.

Since hemostasis processing using plasma can be carried out while directly grasping a blood vessel, a mucous membrane, or the like with the grasping member 110, hemostasis can also be effectively performed to spurting bleeding or an exposed blood vessel, on which conventional plasma had a limited hemostasis effect. In addition, since a blood vessel or the like is directly grasped by the grasping member 110, it is possible to carry out safe hemostasis with little tissue damage. Furthermore, the end effector 100 can be handled in the same manner as a hemostasis clip by having the end effector 100 comprising the grasping member 110 be detachable with respect to the pulling means 170, which enables a certain hemostasis effect to be obtained for a long time. It may be preferable to use the APC mode 13B or the high frequency coagulation mode 13C especially when carrying out hemostasis of a treatment part with high blood pressure using plasma while grasping using the grasping member 110 in such a manner.

FIG. 3 is a cross-section drawing showing the end effector 100 of FIG. 2 in a changed state.

FIG. 3 shows a state after the end effector 100 shifted from an open state to a closed state by an operator pulling the pulling means 170 towards the direction of pulling the end effector 100 inside the insertion part 11. As shown in FIG. 3, the projection part 111a and the projection part 111b are engaged with the concave part 182 when the grasping member 110 is in a closed state. This causes the end effector 100 to not be pulled inside the insertion part 11 further than the position shown in FIG. 3 and causes the grasping member 110 to not shift to an open state. When the operator pulls the pulling means 170 towards the direction of pulling the end effector 100 inside the insertion part 11 further from the state shown in FIG. 3, the pulling means 170 will be removed from the end effector 100 and the distal end part 11′ of the insertion part 11 willbe removed from the lock mechanism 180 because the fitting strength of the convex part of the pulling means 170 and the concave part of the end effector 100 and the fitting strength of the convex part of the distal end part 11′ of the insertion part 11 and the concave part 181 of the lock mechanism 180 would be weaker that the fitting strength of the projection parts 111a, 111b and the concave part 182. This enables sole use of the end effector 100 maintaining a closed state by the lock mechanism 180. Therefore, as discussed above, the end effector 100 can be handled in the same manner as a hemostasis clip.

As shown in FIG. 3, the plasma generation mechanism 120 is configured to enable irradiation of plasma to a position where the grasping member 110 grasps tissue (i.e., a position where the distal end part of the first grasping piece 110a and the distal end part of the second grasping piece 110b are connected to each other). In other words, the end effector 100 can irradiate plasma to tissue using the plasma generation mechanism 120 to treat the tissue in a state in which the tissue is grasped with the grasping member 110. In addition, the grasping member 110 plays a role of a guide mechanism indicating the irradiation direction of plasma by setting the position of the release hole 130 so as to irradiate plasma to the position where the grasping member 110 grasps the tissue. This enables accurate positioning of the irradiation position of plasma. Furthermore, the irradiation position of plasma can be visualized and the irradiation position of plasma can be accurately positioned by providing a laser light source for indicating the irradiation direction and irradiation position of plasma to the end effector 100 or the endoscopic system 10.

The Example shown in FIG. 2 and FIG. 3 explained a case of an embodiment of a connection wherein the distal end part of the pulling means 170 fits with the proximal end part of the end effector 100. However, the present invention is not limited thereto. The embodiment of the connection between the end effector 100 and the pulling means 170 is optional as long as they are detachable from each other. For example, each of the proximal end part of the end effector 100 and the pulling means 170 may have a corresponding screw thread cut thereto, or may be provided with a magnet that enables the end effector 100 and the pulling means 170 to be detachably connected. In addition, the Example shown in FIG. 2 and FIG. 3 explained an embodiment of connection wherein the distal end part 11′ of the insertion part 11 fits with the proximal end part of the lock mechanism 180. However, the present invention is not limited thereto. The embodiment of the connection between the distal end part 11′ of the insertion part 11 and the lock mechanism 180 is also optional as long as they are detachable from each other. For example, each of the distal end part 11′ of the insertion part 11 and the lock mechanism 180 may have a corresponding screw thread cut thereto, or may be provided with a magnet that enables the distal end part 11′ and the lock mechanism 180 to be detachably connected.

In addition, the Example shown in FIG. 2 and FIG. 3 explained that a closed state of the grasping member 110 is maintained by the projection part 111a and projection part 111b and concave part 182. However, the present invention is not limited thereto. The closed state of the grasping member 110 may be maintained by any means that can maintain the closed state of the grasping member 110. For example, there may be a configuration wherein the grasping pieces 110a and 110b comprise a magnet (not shown), wherein when the grasping pieces 110a and 110b are in a closed state within a predetermined range due to the force of the magnet, the closed state is maintained.

In addition, in the example shown in FIG. 2 and FIG. 3, the number of grasping pieces is 2. However, the present invention is not limited thereto. The number of grasping pieces is any number that is 2 or greater. For example, the grasping member 110 may comprise 3 grasping pieces, or may comprise 4 grasping pieces.

In addition, in the example shown in FIG. 2 and FIG. 3, the grasping member 110 is opened/closed with physical contact. However, the configuration of the grasping member 110 is not limited thereto. For example, the grasping member 110 may be configured so that the opening/closing of the grasping member 110 can be electrically controlled with an operation part 12. This enables the operator to open/close the grasping member 110 without pulling the end effector 100 in order to open/close the grasping member 110.

As such, according to the endoscopic system 10 of the present invention, plasma irradiation to tissue, grasping of tissue with the grasping member 110, switching among the low temperature plasma mode, the APC mode and the high frequency coagulation mode and pulse-like gas irradiation can all be practiced with one end effector of the present invention. Conventionally, it was necessary to replace the hemostasis tool (for example, grasping member, APC apparatus, high frequency coagulation apparatus and low temperature plasma apparatus) which is to be inserted in the insertion part of the endoscope each time in accordance with the situation of the bleeding, which thereby caused a therapy to take a long time and caused large burden to a patient.

Meanwhile, according to the endoscopic system 10 comprised by the end effector 100 of the present invention, various hemostasis methods can be selected or combined for therapy by switching the mode or the like in accordance with the situation without going through the trouble of replacing the hemostasis tool, which is thereby significant in terms of being able to accurately perform therapy in a secure/safe and prompt manner.

For example, for gushing bleeding that was unable to be treated with conventional low temperature plasma, the combination of the low temperature plasma and the pulse-like gas irradiation or the grasping member enables hemostasis of the gushing bleeding in high speed and without damaging tissue. In addition, for spurting bleeding that was unable to be treated with conventional low temperature plasma, the combination of the grasping of tissue with the grasping member 110 and the APC or the high frequency coagulation enables hemostasis of the spurting bleeding. Furthermore, hemostasis of an exposed blood vessel can be carried out by utilizing high frequency coagulation.

In addition, it can be considered that the endoscopic system 10 of the present invention which can handle the low temperature plasma and the grasping member 110 is significant in that irradiation of low temperature plasma and grasping of tissue with the grasping member both have high safety.

Although the present invention has been exemplified using a preferable embodiment of the present invention as described above, the interpretation of the present invention should not be limited to this embodiment. It is understood that the scope of the present invention should be interpreted by the Claims alone. It is understood that those skilled in the art can practice an equivalent scope based on the description of the present invention and common general knowledge from the description of the specific and preferable embodiment of the present application.

### INDUSTRIAL APPLICABILITY

The present invention is useful as an invention providing an improved end effector for hemostasis, an endoscopic system comprising the end effector and the like.

### REFERENCE SIGNS LIST

10 endoscopic system
100 end effector
110 grasping member
120 plasma generation mechanism
130 release hole
140 inflow hole
150 electrode
160 hinge part

1. An end effector comprising:
a grasping member for grasping tissue; and
a plasma generation mechanism that can generate plasma.

2. The end effector of claim 1, further comprising a hinge part, wherein:
the grasping member and the plasma generation mechanism are connected to each other at the hinge part; and
the grasping member is configured to be able to rotate around the hinge part.

3. The end effector of claim 1, further comprising a connection part that can connect with a pulling means that can pull the plasma generation mechanism, wherein activation of the pulling means that has been connected achieves grasping of the tissue with the grasping member.

4. The end effector of claim 3, wherein the connection part is configured so that the puling means would be detachable.

5. The end effector of claim 1, wherein the grasping member is configured to be able to be electrically controlled.

6. The end effector of claim 1, wherein the plasma generation mechanism is configured so that the plasma can be irradiated to a position where the grasping member grasps the tissue.

7. The end effector of claim 1, wherein the grasping member comprises a plurality of grasping pieces.

8. The end effector of claim 1, wherein:
the plasma generation configuration has a housing shape having a hollow part;
the housing comprises a first electrode and a second electrode that is different from the first electrode; and
the plasma generation mechanism turns gas that passes through the hollow part into plasma by electric release between the first electrode and the second electrode.

9. An endoscopic system comprising the end effector of claim 1.

10. The endoscopic system of claim 9, further comprising:
a gas supply source that can supply gas that is to be turned into plasma with the plasma generation mechanism, wherein the gas supply source can supply one or more type of gas;
a power source that can switch among a plurality of modes; and
a pulling means that can be connected to the connection part of the end effector.

11. The endoscopic system of claim 10, wherein the plurality of modes include at least two of a low temperature plasma mode, an APC (argon plasma coagulation) mode and a high frequency coagulation mode.

12. The endoscopic system of claim 9, further comprising a pulse gas system for enabling pulse-like supply of gas that is to be turned into plasma to the hollow part.
